(19) United States
(12) Patent Application Publication
(10) Pub. No.: US 2008/0139507 A1
(43) Pub. Date: Jun. 12, 2008

(54) Method of Treating Skin Condition Including Acne, Skin Aging, Body Odor & Diaper Rash by Zinc Zeolite Clathrates

(75) Inventor: SHYAM K. GUPTA, SCOTTSDALE, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, SCOTTSDALE, AZ (US)

(21) Appl. No.: 12/032,751

(22) Filed: Feb. 18, 2008

Related U.S. Application Data
(63) Continuation-in-part of application No. 10/711,136, filed on Aug. 26, 2004.
Application No. 10/711,136 is a continuation-in-part of application No. 11/307,824, filed on Feb. 24, 2006.
Application No. 11/307,824 is a continuation-in-part of application No. 11/684,702, filed on Mar. 12, 2007.
Application No. 11/684,702 is a continuation-in-part of application No. 11/760,466, filed on Jun. 8, 2007.

(51) Int. Cl. A61K 31/695

(52) U.S. Cl. 514/63

(57) ABSTRACT

The present invention discloses a method of treating skin conditions that include acne, skin wrinkles, age spots, pimples, scars, skin rash including diaper rash, dry skin, scalp dandruff, scalp dry skin, darkened skin, skin discoloration, broken skin, chafed skin, sunburn, skin damage from UV, skin irritation, body odor, or a combination thereof, via topical application of certain clathrates of zinc zeolite with non-antibiotic organic agents.

This is a continuation-in-part of U.S. patent application Ser. Nos. 10/711,136 (filed Aug. 26, 2004), 11/307,824 (filed Feb. 24, 2006), 11/684,702 (filed Mar. 12, 2007), and 11/760,466 (filed Jun. 8, 2007).

### BACKGROUND OF THE INVENTION

The present invention discloses a method of treating skin conditions via topical application of certain clathrates of zinc zeolite with non-antibiotic organic agents. This method can include a base, a carrier, or a delivery system. This method provides a combination of topical benefits, which includes alleviation of skin conditions such as acne, skin wrinkles, body odor, skin rash including diaper rash, dry skin, scalp dandruff, darkened skin, broken or chafed skin, sunburn, skin damage from UV, skin irritation, or a combination thereof.

### DESCRIPTION OF THE RELATED ART

Zeolites are a group of crystalline aluminosilicates that have a porous, cage-like structure with a cavity. A zeolite may be defined as an aluminosilicate with a framework structure enclosing cavities occupied by large ions and water molecules, both of which have considerable freedom of movement, permitting ion-exchange and reversible dehydration. The framework consists of an open arrangement of corner sharing tetrahedral where SiO4 are partially replaced by AlO4 tetrahedra, which requires sufficient cations to achieve electro neutrality.

There are some 50 natural and over 150 synthetic zeolites, the latter all made by hydrothermal synthesis. The main uses are as molecular sieves, catalysts, and catalyst support for platinum group metals. Zeolite cavities are usually occupied by water.

Dehydration of synthetic zeolites leaves cubic micro crystals in which AlO4 and SiO4 tetrahedra are linked together to form a ring of eight O atoms on each face of the unit cube and an irregular ring of six O atoms across each corner. In the center of the unit cell is a large cavity about 11.4 Angstroms in diameter, which is connected to six identical cavities in adjacent unit cells by the eight-membered rings, which have inner diameter of about 4.2 Angstroms. In addition, the large cavity is connected to eight smaller cavities, about 6.6 Angstroms in diameter, by the six-membered rings, which provide openings of about 2.0 Angstrom in diameter. In the hydrated form all the cavities contain water molecules. In the anhydrous state the same cavities may be occupied by other molecules brought into contact with the zeolite, provided such molecules are able to pass through the apertures connecting cavities. Molecules within the cavities then tend to be held there by attractive forces of electrostatic and van der Waals types. Thus the zeolites will be able to absorb and strongly retain molecules just small enough to enter the cavities. It will not absorb at all those too big to enter. It will absorb weakly very small molecules that can enter or leave easily, except water molecules, which bind strongly.

The preparation and properties of anionic zeolites are described in detail in U.S. Pat. No. 2,882,243, among other sources. Generally, the preparation involves combining aqueous solutions that are sources of silica, alumina and sodium to produce a gel that crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300 C provides a zeolite that has a strong heat of hydration.

Zeolites can be made with both specific pore structures and bound cations such as Na, K, Mg, Ca, and Zn, that have found applications in various self-warming cosmetic compositions in the prior art. U.S. Pat. No. 3,250,680 (Menkart et al.) discloses applications of Zeolites for the preparation of self-heating toothpaste and other such compositions. Menkart utilizes only the heat releasing property of zeolites.

The inorganic complexes of zeolites are well known, for example, RU 2163510 discloses method for fixing metal complexes by the aid of T-shaped anchoring fragment in large cavities of zeolites. U.S. patent application Ser. No. 2007000382 discloses silver trapped zeolite complexes. DE 19913395 discloses certain inclusion compounds (I) based on a zeolite host lattice comprise metal clusters, transition metal complexes, noble metal complexes and dyes contained in mesopores of a zeolite, the mesopores being enclosed exclusively by micropores. U.S. Pat. No. 5,429,814 disclose a method of using molecular sieve-enclosed paramagnetic ions as image brightening or image contrast agents. In particular, zeolite enclosed trivalent gadolinium is useful in MRI studies of the entire gastrointestinal tract. U.S. Pat. No. 4,472,517 discloses a method of incorporating metals onto a crystalline aluminosilicate zeolite support comprises first depositing a metal which forms bonds with the zeolite and subsequently depositing a catalytically active metal into the zeolite which becomes associated with the first metal. Although it is not meant to serve as an exhaustive prior art summary of this field, it is surprising that organic clathrate complexes of zeolites, especially zinc zeolites, are not known to the same extent as their inorganic counterparts.

U.S. patent application Ser. No. 11/307,824 (filed on Feb. 24, 2006) by the present inventor disclosed the application of certain cage complexes of certain alkali and alkaline earth metal zeolites for topical controlled delivery of certain organic cosmetic and pharmaceutical active agents. A method for the treatment of any skin conditions via topical application of said cage complexes was not disclosed. The present invention discloses a method for treating skin conditions that include acne, skin wrinkles, skin rash including diaper rash, dry skin, scalp dandruff, darkened skin, broken skin, chafed skin, sunburn, skin damage from UV, skin irritation, or a combination thereof, via topical application of certain clathrates of zinc zeolite with non-antibiotic organic agents.

Moreover, it is worthy of note that, both surprisingly and unexpectedly, the alkali and alkaline earth metal zeolites, discussed in U.S. patent application Ser. No. 11/307,824, do not form stable clathrate complexes with non-antibiotic organic compounds or agents, while the clathrates of zinc zeolite with non-antibiotic organic compounds or agents, as discovered in the present invention, are quite stable and amenable to various compositions traditionally used in cosmetic and pharmaceutical preparations. While it was the relative instability of cage complexes disclosed in U.S. patent application Ser. No. 11/307,824 that permitted the controlled release of active agents from said cage complexes, the relative stability of clathrate complexes of the present invention provides an unexpected and surprising method for the treatment of various topical disorders, which are disclosed herein. The reason for this change of stability properties from alkali and alkaline earth metal zeolites to zinc zeolites is not known at this time, although the stability offered by the chelating properties of zinc atom, via its d-orbital as illustrated in FIGS. 1-3, may not be possible with alkali and alkaline earth metal zeolites. However, this lack of precise knowledge should not alter the importance or utility of the present invention.

U.S. patent application Ser. No. 11/684,702 (filed Mar. 12, 2007) by the present inventor disclosed certain complexes of anionic zeolites, which did not include zinc zeolites, with certain active oxygen agents such as organic and inorganic peroxides, the incorporation of said complexes in certain topical compositions, and the application of said complexes and said compositions for skin whitening, and skin discoloration and age spot reduction.

JP 2007197341 and JP2007196104 (Masuda et al.) disclose an antimicrobial agent that comprises zeolite, an oxygen-activating metal complex encapsulated in the unit cells of the zeolite, and a photo-reductive metal complex held in the zeolite (preferably encapsulated in the unit cells of the zeolite). Preferably, the zeolite contains a metal (for example, one or more metals selected from silver, copper and zinc) belonging to the group 1 to 13 in the periodic table. The oxygen-activating metal complex includes a metal phthalocyanine complex and a metal salophen complex synthesized in a unit cell by ship-in-bottle method. Masuda et al. do not disclose any organic non-antimicrobial agent complexes of zinc zeolites with said agents, and wherein said complexes are non-antimicrobial themselves but still impart topical benefits such as the treatment for acne that are not based on antimicrobial action of said complexes.

WO 02100420 discloses a pharmaceutical composition, which includes a synergic association of erythromycin and a carrier based on zeolite brought to Zn form. The preparation process is also described, which is based on an initial exchange of the Na ions present in the zeolite with Zn; this zeolite in zinc form is then mixed with erythromycin. However, it is should be noted that erythromycin, in itself, is well known to form complexes with many other molecules, for example, Mirza et al., “Influence of Solvents on the Variety of Crystalline Forms of Erythromycin”, AAPS Pharm. Sci. 2003; 5 (2): article 12, pages 1-9. Azithromycin, a chemical structural analog of erythromycin, is also known to form clathrates (U.S. Pat. Nos. 7,235,646; 6,245,903). It is thus not surprising or unexpected that erythromycin, similar to many other antibiotics, forms complexes with zeolites, including zinc zeolite. The combinations of zinc zeolite with any non-antibiotic agents were not disclosed by Mirza et al. Also, erythromycin is a well-known antibiotic agent used for the treatment of acne and any combination of erythromycin with zinc zeolite would also be expected to be useful for the treatment of acne, which would additionally render the disclosures in WO 02100420 of not surprising but of quite expected nature. For these reasons, zinc zeolite antibiotic clathrates have been excluded from the claims of the present invention, and only zinc zeolite non-antibiotic clathrates have been disclosed.

U.S. Pat. Nos. 4,911,899 and 4,911,898 (Hagiwara et al.) disclose certain bactericidal zinc and silver zeolites, which do not include any non-antibacterial organic agents.

Benzaminson et al. (WO 2006098680) disclose the use of a hydrophobic zeolite, that contains an active component, especially a disinfection element, as ethanol, iodine, phenol, cresol or hydrogen peroxide, in a composition for non-medical treatment of the skin, for example as a deodorant. The invention also describes the use of a hydrophobic zeolite, that contains an active component, especially a disinfection element, as hydrogen peroxide, for manufacturing of a pharmaceutical preparation for treatment of skin related conditions and diseases, as skin infections. The hydrophobic zeolite is especially selected from that group that comprises silicalite, or hydrophobic ZMS-5, hydrophobic mordenite and hydrophobic zeolite Y.

U.S. Pat. No. 5,476,660 (Somasundaran et al.) discloses certain compositions of chemically modified zeolites in which zeolite surface has been modified to a positively charged state (cationic) or a zwitterionic state. These chemically modified zeolites have a filamentous structure with outwardly protruding positively charged organocarbonyl groups and also outwardly protruding negatively charged organocarbonyl groups. These chemically modified zeolites are useful for the deposition of active agents, more specifically, anionic active agents.

U.S. Pat. No. 4,626,550 (Hertzenberg) discloses certain personal care products such as lotions and creams that are prepared using potassium exchanged Zeolite A that is much less anionic in nature. These compositions are useful only for the release of heat, and the inclusion of active agents such as bodying agents, topical pain relievers, antiperspirants and others must be largely anhydrous and should not enter the structures of the zeolite to release heat (col. 3, line 50-57). Hertzenberg does not disclose any zinc zeolite based clathrate complexes or a method of topical treatment with said clathrates.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems. Varying the character of the liquid vehicle can control generation and maintenance of the heat of hydration of anhydrous zeolite. If a very quick release of heat is desired, a hydrophilic vehicle is used; if a slow, sustained heat release is desired, a hydrophobic vehicle is required. Intermediate and controlled performance can be introduced by altering the hydrophobic vehicle to provide some hydrophilic characteristics. Sherry et al. do not disclose any zinc zeolite based clathrate complexes or a method of topical treatment with said clathrates.

U.S. Pat. No. 6,274,128 (Bergman et al.) discloses an essentially anhydrous hair conditioning composition that comprises zeolites of specific pore size larger than the critical diameter of a water molecule and both the carrier molecules and the hair conditioner molecules that have molecular diameters larger than the largest average pore size of the micro porous materials. Bergman et al. utilize only the heat-releasing or rubefacient properties of zeolites and do not disclose any zinc zeolite based clathrate complexes or a method of topical treatment with said clathrates.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect. Minnix utilizes only the heat-releasing or rubefacient properties of zeolites and does not disclose any zinc zeolite based clathrate complexes or a method of topical treatment with said clathrates.

U.S. Application 20010016201 (Janchitraponvej) discloses a yet another self-heating application of an anhydrous rinse-out hair care composition utilizing zeolites.

Self-warming compositions have also been made with various anhydrous alkali metal salts (Giani et al., U.S. Pat. No. 5,747,004). In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. Most cosmetic lotion, cream, shampoo, and conditioner products also contain hydrophilic and lipophilic ingredients for skin and hair care benefits. Some of such ingredients tend to clog the pores of Zeolites, causing a reduction in the heat-release properties of such formulations. The examples of such fatty materials that can inhibit the heat release properties of zeolites include most surfactants used in shampoo and body wash applications; quaternary ammonium compounds used for hair conditioning applications; fatty esters used as emollients in skin lotion and cream applications, and other similar examples. While such clogging of zeolite pores by above mentioned ingredients, some of which are highly desirable active agents, was considered a problem, and those problems were solved in the prior art by the use of small pore size zeolites that permit the entrance of water molecules inside their cavity but not other larger size molecules, for example U.S. Pat. No. 6,274,128.

U.S. patent application Ser. No. 20050133049 (Fournier et al.) discloses filters, smoking articles, and methods for selectively removing one or more selected constituents from mainstream smoke. The filters comprise zeolite BETA. Fournier et al. did discover that certain organic agents can bind with zeolite, but they did not disclose any zinc zeolite based clathrate complexes or a method of topical treatment with said clathrates.

U.S. patent application Ser. No. 20050058597 (Corbin et al.) discloses a process to synthesize nano-size Zeolite A from an amorphous gel precursor. The nano-sized Zeolite A has been used for detergents. Corbin et al. did not disclose any zinc zeolite based clathrate complexes or a method of topical treatment with said clathrates.

It is worthy of note that although zeolites with many different cations, such as titanium, zinc, manganese, iron, and copper have been disclosed, any applications of such metal zeolites in any zinc zeolite based clathrate complexes or a method of topical treatment with said clathrates has not been disclosed.

U.S. Pat. No. 6,503,740 (Alther et al.) discloses zeolites treated with an organic modification compound such as quaternary amines, pyridinium compounds, and phosphonium amines that are useful for water treatment applications. Alther et al. do not disclose any zinc zeolite based clathrate complexes or a method of topical treatment with said clathrates.

U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman). However, none of these disclose any zinc zeolite based clathrate complexes or a method of topical treatment with said clathrates.

### BRIEF SUMMARY OF THE INVENTION

The present invention discloses a method of treating skin conditions that include acne, skin wrinkles, age spots, pimples, scars, skin rash including diaper rash, dry skin, scalp dandruff, scalp dry skin, darkened skin, skin discoloration, broken skin, chafed skin, sunburn, skin damage from UV, skin irritation, body odor, or a combination thereof, via topical application of certain clathrates of zinc zeolite with non-antibiotic organic agents.

### BRIEF DESCRIPTION OF THE SEVERAL VIEWS OF THE DRAWINGS

FIG. 1 Zinc Zeolite—Salicylate Clathrate Complex.

FIG. 2 Zinc Zeolite—Ethylhexylglycerin Clathrate Complex.

FIG. 3 Zinc Zeolite—Lactate Clathrate Complex

### DETAILED DESCRIPTION

The present invention discloses a method for treating skin condition, wherein (i) a clathrate complex of zinc zeolite is applied topically at a desired site in a sufficient quantity, and (ii) wherein said application having been done either by a manual or a mechanical mean, or a combination thereof, and (iii) wherein said topical application causes the desired treatment of said skin condition.

In the present invention, said clathrate complex of zinc zeolite is of the chemical composition consisting of zinc zeolite and a non-antibiotic organic compound or agent. Any clathrates of zinc zeolite and an antibiotic organic compound are excluded.

The clathrate complexes are chemical substances consisting of a lattice of one kind of molecule or agent trapping and containing a second type of molecule or agent. These are also called host-guest complexes, in which one molecule acts as a host, trapping or containing a guest molecule. The clathrates are of various nature, and include, for example, strong interaction between host molecules and guest molecules, or guest molecules set in geometrical spaces of host molecule by weak intermolecular forces.

In the treatment method of the present invention, said skin condition is selected from the group consisting of acne, skin wrinkles, skin rash including diaper rash, dry skin, scalp dandruff, darkened skin, broken or chafed skin, sunburn, skin damage from UV, skin irritation, or a combination thereof.

In the treatment method of the present invention, said clathrate complex of zinc zeolite is further selected from the group consisting of zinc zeolite hydroxy acids, zinc zeolite hydroxy acid esters, zinc zeolite hydroxy acid salts, zinc zeolite organic peroxides, zinc zeolite organic hydroperoxides, zinc zeolite lignans, zinc zeolite enzyme inhibitors, zinc zeolite vitamins, zinc zeolite hormones, zinc zeolite peptides, zinc zeolite polyphenols, zinc zeolite stilbenes, zinc zeolite alkaloids, zinc zeolite plant extracts, zinc zeolite antioxidants, zinc zeolite glycols and polyglycols, zinc zeolite sunscreens, zinc zeolite saponins, zinc zeolite anesthetics, zinc zeolite sapogenins, zinc zeolite triterpenes, zinc zeolite amino acids, or combinations thereof.

In the treatment method of the present invention, said clathrate complex of zinc zeolite hydroxy acids, their esters, and salts are further selected from, but not limited to alpha, beta, and polyhydroxy acids, their esters and salts.

The zinc zeolite hydroxy acids and their salts are further selected from, but not limited to, zinc zeolite clathrates of 2-hydroxyethanoic acid (glycolic acid), 2-hydroxypropanoic acid (lactic acid), 2-methyl 2-hydroxypropanoic acid (methyl lactic acid), 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxyeicosanoic acid (alpha hydroxyarachidonic acid), 2-hydroxytetraeicosanoic acid (cerebronic acid), 2-hydroxytetraeicosenoic acid (alpha hydroxynervonic acid), 2,4-dihydroxy-3,3-dimethylbutanoic acid (pantoic acid), 2-phenyl 2-hydroxyethanoic acid (mandelic acid); 2,2-diphenyl 2-hydroxyethanoic acid (benzilic acid), 3-phenyl 2-hydroxypropanoic acid (phenyl)acetic acid), 2-phenyl 2-methyl 2-hydroxyethanoic acid (atrolactic acid), 4-hydroxymandelic acid, 2,3-dihydroxypropanoic acid (glyceric acid); 2,3,4-trihydroxybutanoic acid (isomers; erythronic acid, threonic acid); 2,3,4,5-tetrahydroxypentanoic acid (isomers; ribonic acid, arabinoic acid, xylonic acid, lyxonic acid); 2,3,4,5,6-pentahydroxyhexanoic acid (isomers; allonic acid, altronic acid, gluconic acid, mannoic acid, gulonic acid, idonic acid, galactonic acid, talonic acid); 2,3,4,5,6,7-hexahydroxyheptanoic acid, 2-hydroxypropane-1,3-dioic acid (tartronic acid); 2-hydroxybutane-1,4-dioic acid (malic acid); 2-hydroxy-2-methylbutane-1,4-dioic acid (citramalic acid); 2,3-dihydroxybutane-1,4-dioic acid (tartaric acid); 2,3,4-trihydroxypentane-1,5-dioic acid (isomers; ribaric acid, arabaric acid, xylaric acid, lyxaric acid); 2,3,4,5-tetrahydroxyhexane-, 1,6-dioic acid (isomers; glucaric acid, galactaric acid, mannaric acid, allaric acid, altraric acid, gularic acid, idaric acid, talaric acid); 2-hydroxy-1,2,3-propanetricarboxylic acid (citric acid); Hydroxycitric acid, Garcinia Acid, 1-hydroxy-1,2,3-propanetricarboxylic acid (isocitric acid); 1-hydroxy-1,2,4-butanetricarboxylic acid (homoisocitric acid); 2-hydroxy-3-hexadecyl-1,2,3-propanetricarboxylic acid, glyceruronic acid, erythruronic acid, threuronic acid; 2,3,4-trihydroxypentanuronic acids (isomers; riburonic acid, arabinuronic acid, xyluronic acid, lyxuronic acid); 2,3,4,5-tetrahydroxyhexanuronic acid (isomers; alluronic acid, altruronic acid, glucuronic acid, mannuronic acid, guluronic acid, iduronic acid, galacturonic acid, taluronic acid), and 2,3,4,5,6-pentahydroxyheptanuronic acid (isomers; alloheptanuronic acid, altroheptanuronic acid, glucoheptanuronic acid, mannoheptanuronic acid, guloheptanuronic acid, idoheptanuronic acid, galactoheptanuronic acid, taloheptanuronic acid, and the corresponding zinc salts of these acids, and combinations thereof.

The zinc zeolite beta hydroxy acids and their salts are selected from salicylic acid and zinc salicylate.

The clathrates of zinc zeolite hydroxy esters are selected from, but not limited to zinc zeolite clathrate complexes of an ester of a hydroxy acid selected from the group consisting of:

the methyl, ethyl, propyl, isopropyl, butyl, pentyl, octyl, decyl and dodecyl esters of glycolic acid;
the methyl, ethyl, propyl, isopropyl, butyl, pentyl, octyl, decyl and dodecyl esters of lactic acid;
the methyl, ethyl, propyl, isopropyl, butyl, pentyl, octyl, decyl and dodecyl esters of methyl lactic acid;
the methyl, ethyl, propyl, isopropyl, butyl, pentyl, octyl, decyl and dodecyl esters of 2-hydroxybutanoic acid;
the methyl, ethyl, propyl, isopropyl, butyl, pentyl, octyl, decyl and dodecyl esters of 2-hydroxypentanoic acid; the methyl, ethyl, propyl, isopropyl, butyl, pentyl, octyl, decyl and dodecyl esters of 2-hydroxyhexanoic acid;
the methyl, ethyl, propyl, isopropyl, butyl, pentyl, octyl, decyl and dodecyl esters of 2-hydroxyheptanoic acid;
the methyl, ethyl, propyl, isopropyl, butyl, pentyl, octyl, decyl and dodecyl esters of 2-hydroxyoctanoic acid; the methyl, ethyl, propyl, isopropyl, butyl, pentyl, octyl, decyl and dodecyl esters of 2-hydroxynonanoic acid;
the methyl, ethyl, propyl, isopropyl, butyl, pentyl, octyl, decyl and dodecyl esters of 2-hydroxydecanoic acid;
the methyl, ethyl, propyl, isopropyl, butyl, pentyl, octyl, decyl and dodecyl esters of 2-hydroxyundecanoic acid;
the methyl, ethyl, propyl, isopropyl, butyl, pentyl, octyl, decyl and dodecyl esters of 2-hydroxydodecanoic acid;
the methyl, ethyl, propyl, isopropyl, butyl, pentyl, octyl, decyl and dodecyl esters of 2-hydroxytetradecanoic acid;
the methyl, ethyl, propyl, isopropyl, butyl, pentyl, octyl, decyl and dodecyl esters of 2-hydroxyhexadecanoic acid;
the methyl, ethyl, propyl, isopropyl, butyl, pentyl, octyl, decyl and dodecyl esters of 2-hydroxyoctadecanoic acid;
the methyl, ethyl, propyl, isopropyl, butyl, pentyl, octyl, decyl and dodecyl esters of 2-hydroxyeicosanoic acid;
the methyl, ethyl, propyl, isopropyl, butyl, pentyl, octyl, decyl and dodecyl esters of 2-hydroxytetraeicosanoic acid; and
the methyl, ethyl, propyl, isopropyl, butyl, pentyl, octyl, decyl and dodecyl esters of 2-hydroxytetraeicosenoic acid, the corresponding acids of these esters, and the corresponding zinc salts of those acids, and combinations thereof.

The zinc zeolite poly hydroxy acids, their esters, and salts are selected from clathrate complexes of zinc zeolite and any of the methyl, ethyl, propyl, isopropyl, butyl, pentyl, octyl, decyl and dodecyl esters of 2-phenyl 2-hydroxyethanoic acid esters;

the methyl, ethyl, propyl, isopropyl, butyl, pentyl, octyl, decyl and dodecyl esters of 2,2-diphenyl 2-hydroxyethanoic acid;
the methyl, ethyl, propyl, isopropyl, butyl, pentyl, octyl, decyl and dodecyl esters of 3-phenyl 2-hydroxypropanoic acid; and
the methyl, ethyl, propyl, isopropyl, butyl, pentyl, octyl, decyl and dodecyl esters of 2-phenyl 2-methyl 2-hydroxyethanoic acid, the methyl, ethyl, propyl, isopropyl, butyl, pentyl, octyl, decyl, dodecyl, phenyl and benzyl esters of 2,3-dihydroxypropanoic acid;
the methyl, ethyl, propyl, isopropyl, butyl, pentyl, octyl, decyl, dodecyl, phenyl and benzyl esters of 2,3,4-trihydroxybutanoic acid and its isomers including erythronic acid and threonic acid; the methyl, ethyl, propyl, isopropyl, butyl, pentyl, octyl, decyl, dodecyl, phenyl and benzyl esters of 2,3,4,5-tetrahydroxypentanoic acid and its isomers including ribonic acid, arabinoic acid, xylonic acid and lyxonic acid;
the methyl, ethyl, propyl, isopropyl, butyl, pentyl, octyl, decyl, dodecyl, phenyl and benzyl esters of 2,3,4,5,6-pentahydroxyhexanoic acid and its isomers including allonic acid, altronic acid, gluconic acid, mannoic acid, gulonic acid, idonic acid, galactonic acid, and talonic acid; the methyl, ethyl, propyl, isopropyl, butyl, pentyl, octyl, decyl, dodecyl, phenyl and benzyl esters of 2,3,4,5,6,7-hexahydroxyheptanoic acid and its isomers including glucoheptonic acid and galactoheptonic acid;
the methyl, ethyl, propyl, isopropyl, butyl, pentyl, octyl, decyl, dodecyl, phenyl and benzyl esters of glyceruronic acid;
the methyl, ethyl, propyl, isopropyl, butyl, pentyl, octyl, decyl, dodecyl, phenyl and benzyl esters of erythruronic acid;
the methyl, ethyl, propyl, isopropyl, butyl, pentyl, octyl, decyl, dodecyl, phenyl and benzyl esters of threuronic acid;
the methyl, ethyl, propyl, isopropyl, butyl, pentyl, octyl, decyl, dodecyl, phenyl and benzyl esters of riburonic acid;
the methyl, ethyl, propyl, isopropyl, butyl, pentyl, octyl, decyl, dodecyl, phenyl and benzyl esters of arabinuronic acid;
the methyl, ethyl, propyl, isopropyl, butyl, pentyl, octyl, decyl, dodecyl, phenyl and benzyl esters of xyluronic acid;
the methyl, ethyl, propyl, isopropyl, butyl, pentyl, octyl, decyl, dodecyl, phenyl and benzyl esters of lyxuronic acid;
the methyl, ethyl, propyl, isopropyl, butyl, pentyl, octyl, decyl, dodecyl, phenyl and benzyl esters of alluronic acid;
the methyl, ethyl, propyl, isopropyl, butyl, pentyl, octyl, decyl, dodecyl, phenyl and benzyl esters of altruronic acid;
the methyl, ethyl, propyl, isopropyl, butyl, pentyl, octyl, decyl, dodecyl, phenyl and benzyl esters of glucuronic acid;
the methyl, ethyl, propyl, isopropyl, butyl, pentyl, octyl, decyl, dodecyl, phenyl and benzyl esters of mannuronic acid;
the methyl, ethyl, propyl, isopropyl, butyl, pentyl, octyl, decyl, dodecyl, phenyl and benzyl esters of guluronic acid;
the methyl, ethyl, propyl, isopropyl, butyl, pentyl, octyl, decyl, dodecyl, phenyl and benzyl esters of iduronic acid;
the methyl, ethyl, propyl, isopropyl, butyl, pentyl, octyl, decyl, dodecyl, phenyl and benzyl esters of galacturonic acid; and
the methyl, ethyl, propyl, isopropyl, butyl, pentyl, octyl, decyl, dodecyl, phenyl and benzyl esters of taluronic acid, and combinations thereof.

In the treatment method of the present invention, said clathrate complex of zinc zeolite organic peroxides and hydroperoxides are further selected from, but not limited to zinc zeolite benzoyl peroxide, zinc zeolite artemisinin, zinc zeolite artemisia annua extract, and zinc zeolite benzoyl hydroperoxide.

In the treatment method of the present invention, said clathrate complex of zinc zeolite lignans are further selected from, but not limited to zinc zeolite clathrates of silybin, silymarin, silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, nortrachelogenin; their analogs and derivatives, and combinations thereof.

In the treatment method of the present invention, said clathrate complex of zinc zeolite saponins and sapogenins are further selected from, but not limited to zinc zeolite clathrates of Dioscin, Diosgenin, Hecogenin, Heconin, Tigogenin, Tigonin, Gitogenin, Chlorogenin, Eruboside, Protoeruboside, Manogenin, Shlorogenin, Hainangenin, Protodioscin, Protodiosgenin, Aculeoside, Smilagenin, Sarsapogenin, Yamogenin, Yuccagenin, Gracillin, Sativoside, and combinations thereof.

In the treatment method of the present invention, said clathrate complex of zinc zeolite enzyme inhibitor includes zinc zeolite matrix metalloprotease inhibitor, zinc zeolite tyrosinase inhibitor, zinc zeolite superoxide dismutase inhibitor, zinc zeolite 5-alpha reductase inhibitor, zinc zeolite Tumor Necrosis Factor (TNF)-alpha inhibitor, zinc zeolite Ubiquitin-Proteasome inhibitor, zinc zeolite advanced glycation end product inhibitor, zinc zeolite citrate lyase inhibitor, zinc zeolite fatty acid desaturase inhibitor, zinc zeolite urocanate inhibitor, and zinc zeolite prostaglandin-leukotriene pathway inhibitor, and combinations thereof.

In the treatment method of the present invention, said clathrate complexes of zinc zeolite matrix metalloprotease inhibitors are further selected from, but not limited to zinc zeolite clathrates of 2-hydroxyacetophenone, 3-hydroxyacetophenone, 4-hydroxyacetophenone, 2,3-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, Quinacetophenone, 1-(3-Hydroxy-4-methoxy-5-methylphenyl)ethanone, 1-(3-hydroxy-4-methoxyphenyl)ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, 2-hydroxypropiophenone, 3-hydroxypropiophenone, 4-hydroxypropiophenone, 2,3-dihydroxypropiophenone, 2,4-dihydroxypropiophenone, 2,5-dihydroxypropiophenone, 2,6-dihydroxypropiophenone, 3,4-dihydroxypropiophenone, 3,5-dihydroxypropiophenone, 2,4,6-trihydroxypropiophenone, 2,3,4-trihydroxypropiophenone, 2,3,5-trihydroxypropiophenone, 2,3,6-trihydroxypropiophenone, 2,4,5-trihydroxypropiophenone, 3,4,5-trihydroxypropiophenone, 1-(2,4-dihydroxyphenyl)-2-hydroxyethanone, (2-hydroxyphenyl)(oxo)acetic acid, 1-(2,6-dihydroxyphenyl)-1-butanone, 1-(1-hydroxy-2-naphthyl)ethanone, 1-(2-hydroxy-1-naphthyl)ethanone, 5,7-dihydroxy-1-indanone, 1-(2-hydroxy-5-methylphenyl)-1,3-butanedione, N-(4-acetyl-3-hydroxyphenyl)acetamide, 4-acetyl-3-hydroxyphenyl acetate, 1,1′-(4,6-Dihydroxy-1,3-phenylene)bisethanone, 1-(1-hydroxy-2-naphthyl)ethanone, 2,3-Dihydro-9,10-dihydroxy-1,4-anthracenedione, phloridzin, phloretin, and combinations thereof.

In the treatment method of the present invention, said clathrate complexes of zinc zeolite citrate lyase inhibitors are further selected from, but not limited to zinc zeolite clathrates of hydroxycitric acid and zinc hydroxycitrate.

In the treatment method of the present invention, said clathrate complex of zinc zeolite tyrosinase inhibitors are further selected from, but not limited to zinc zeolite clathrates of hydroquinone, arbutin, kojic acid, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, 2-hydroxyacetophenone, 3-hydroxyacetophenone, 4-hydroxyacetophenone, 2,3-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 3,4-Dihydroxyacetophenone, Quinacetophenone, 1-(3-Hydroxy-4-methoxy-5-methylphenyl)ethanone, 1-(3-hydroxy-4-methoxyphenyl)ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, 2-hydroxypropiophenone, 3-hydroxypropiophenone, 4-hydroxypropiophenone, 2,3-dihydroxypropiophenone, 2,4-dihydroxypropiophenone, 2,5-dihydroxypropiophenone, 2,6-dihydroxypropiophenone, 3,4-dihydroxypropiophenone, 3,5-dihydroxypropiophenone, 2,4,6-trihydroxypropiophenone, 2,3,4-trihydroxypropiophenone, 2,3,5-trihydroxypropiophenone, 2,3,6-trihydroxypropiophenone, 2,4,5-trihydroxypropiophenone, 3,4,5-trihydroxypropiophenone, Phloridzin, phloretin, and combinations thereof.

In the treatment method of the present invention, said clathrate complex of zinc zeolite triterpenes are further selected from, but not limited to zinc zeolite clathrates of Asiaticoside (Centella asiatica extract), Boswellia Extract (Boswellia serrata), Sericoside, Visnadine, Thiocolchicoside, Glycyrrhetinic acid, Ursolic acid, Sericoside (Terminalia sericea extract), Darutoside (Siegesbeckia orientalis extract), and combinations thereof.

In the treatment method of the present invention, said clathrate complex of zinc zeolite sunscreen agents are further selected from, but not limited to clathrates of Kaempferia galanga extract, para-Aminobenzoic acid (PABA), Avobenzone, 3-Benzylidene camphor, Benzylidene camphor sulfonic acid, Bisymydazilate, Camphor Benzalkonium Methosulfate, Polyquaternium-59, Cinnamidipropyltrimonium chloride, Diethylamino hydroxybenzoyl hexyl benzoate, Diethylhexyl butamido triazone, Dimethicodiethylbenzal malonate, Drometrizole trisiloxane, Ecamsule, Ensulizole, Homosalate, Isoamyl p-methoxycinnamate, 4-Methylbenzylidene camphor, Octocrylene, Octyl Dimethyl PABA, Cinoxate, Dioxybenzone, Octyl methoxycinnamate, Octyl salicylate, Octyl triazone, Oxybenzone, Polyethylene glycol (PEG)-25 PABA, Polyacrylamidomethyl benzylidene camphor, Sulisobenzone, Methyl anthranilate, Trolamine salicylate, Benzophenone-3, Benzophenone-4, Tinosorb M, Tinosorb S, and combinations thereof.

In the treatment method of the present invention, said clathrate complex of zinc zeolite stilbenes are further selected from, but not limited to zinc zeolite rhapontin, zinc zeolite polydatin, zinc zeolite resveratrol, and combinations thereof.

In the treatment method of the present invention, said clathrate complex of zinc zeolite polyphenols are further selected from, but not limited to zinc zeolite magnolol, zinc zeolite honokiol, zinc zeolite ellagic acid, zinc zeolite hypericin, zinc zeolite tetrahydrocurcumin, zinc zeolite mulberrin, zinc zeolite rosmarinic acid, zinc zeolite chlorogenic acid, zinc zeolite licoricidin, zinc zeolite mangostin, zinc zeolite shikonin, zinc zeolite anhydroalkanin, zinc zeolite glycyrol, zinc zeolite isoliquiritin, zinc zeolite kuraridin, zinc zeolite curcumin, zinc zeolite hydroquinone, zinc zeolite catechol, and combinations thereof.

In the treatment method of the present invention, said clathrate complex of zinc zeolite glycols and polyglycols are further selected from, but not limited to zinc zeolite clathrates of propylene glycol, butylene glycol, pentylene glycol, hexylene glycol, polyethylene glycol, polypropylene glycol, glycerin, diglycerin, polyglycerin, sorbitol, mannitol, sucrose esters, polysorbates, mono-, di- and tri-ethylene glycol monoalkyl ethers, methylpropanediol, ethylhexylglycerin, and combinations thereof.

In the treatment method of the present invention, said clathrate complex of zinc zeolite amino acids are further selected from, but not limited to zinc zeolite clathrates of glycine, alanine, beta-alanine, valine, leucine, isoleucine, phenylalanine, alpha-amino butyric acid, C-phenylglycine, C-hydroxyphenylglycine, proline, tryptophane, lysine, ornithine, arginine, histidine, citrulline, glutamic acid, aspartic acid, serine, threonine, hydroxyproline, tyrosine, dihydroxytyrosine, cysteine, cystine, methionine, homocysteine, lanthionine, or combinations thereof.

In the treatment method of the present invention, said clathrate complex of zinc zeolite peptides are further selected from, but not limited to zinc zeolite clathrates of a large number of dipeptides, tripeptides, tetrapeptides, pentapeptides, hexapeptides, heptapeptides, octapeptides, nonapeptides, and such, including insulin, bradykinin, glutathione, and carnosine as examples. Pickart et al. have disclosed a number of other such peptides (U.S. Pat. Nos. 5,858,993; 5,888,522; 5,698,184; 5,550,183; 5,554,375; 5,164,367; 4,665,054; 4,760,051; 4,810,693 and 4,877,770; U.S. patent application Ser. No. 20050276766), which can be further utilized in the present invention.

In the treatment method of the present invention, said clathrate complex of zinc zeolite vitamins are further selected from, but not limited to zinc zeolite clathrates of Vitamin A, Retinol, Retinoic acid, Tretinoin, members of Vitamins B group, Vitamin C, Vitamin D, Vitamin E, Vitamin K, Carotenes, Biotin, Folic Acid, and their derivatives, and combinations thereof.

In the treatment method of the present invention, said clathrate complex of zinc zeolite hormones are further selected from, but not limited to zinc zeolite clathrates of progesterone, androsterone, dehydroepiandrosterone (DHEA), Pregnenolone, androstenedione, melatonin, testosterone, and combinations thereof.

In the treatment method of the present invention, a clathrate complex of zinc zeolite is applied topically at a desired site in a sufficient quantity, and wherein said application can be done either by a manual or a mechanical methods or a combination thereof. Among mechanical methods, electrically driven instruments, such as rotating or vibrating disks, rotating or vibrating pads, and such, are included. Among manual methods, use of hands or fingers, spatulas, spoons, and such, are included.

However, it is to be noted that it is not the intention to include each and every possible specific example of various chemical groups or classes of clathrate complexes of zinc zeolites or methods of application mentioned above.

The clathrate complex of zinc zeolite and a non-antibiotic organic compound or agent can be made, among other processes, by combining zinc zeolite (host molecule) with said non-antibiotic organic compound or agent (guest molecule) either under anhydrous or hydrous conditions, and either in the presence or absence of a carrier or penetration-enhancing agent. This is shown in [Equation 1].

Zinc Zeolite (Host Molecule)+Non-Antibiotic Organic Compound (Guest Molecule)=Zinc Zeolite−Non Antibiotic Organic Compound (Clathrate Complex) [Equation 1]

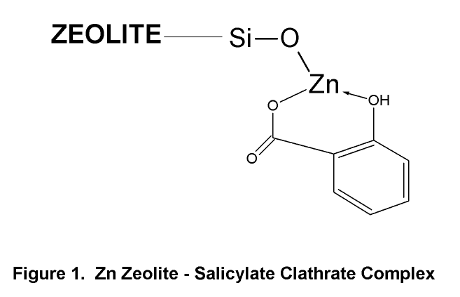

The clathrate complex of zinc zeolite and a non-antibiotic organic compound or agent can have a complex chemical structure, some of which are not yet known. As an example, chemical structure of the clathrate complex of Zinc zeolite salicylate is represented in [FIG. 1]. However, this chemical structure has not been precisely established at this time. This, however, should not make the importance of the present invention any less important or useful.

[FIG. 1].

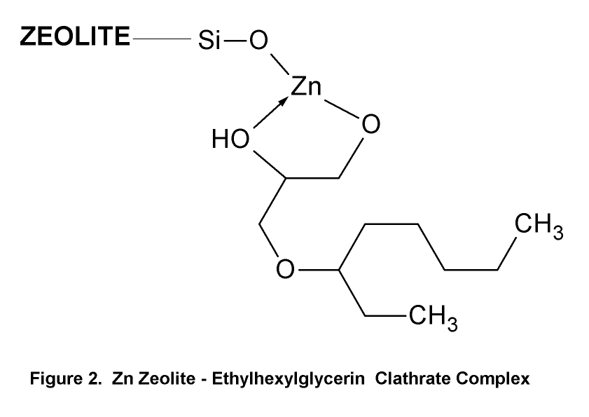

As an additional example, the chemical structure of the clathrate complex of a Zinc zeolite polyglycol, such as zinc zeolite ethylhexylglycerin, is represented in [FIG. 2]. However, this chemical structure has not been precisely established at this time. This, however, should not make the importance of the present invention any less important or useful.

[FIG. 2].

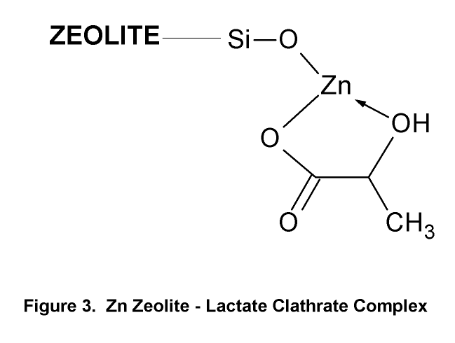

As a final example, the chemical structure of the clathrate complex of Zinc zeolite lactate is represented in [FIG. 3]. However, this chemical structure has not been precisely established at this time. This, however, should not make the importance of the present invention any less important or useful.

[FIG. 3].

The chemical structure of clathrate complexes of zinc zeolite and certain enzyme inhibitors, for example, is yet unknown. However, this chemical structure has not been precisely established at this time. This, however, should not make the importance of the present invention any less meaningful.

In the treatment method of the present invention a variety of delivery systems and carrier base forms can be utilized. Such forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system or a carrier base in the treatment method of the present are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, skin penetration enhancing agent, impregnated or coated diaper, and similar delivery or packaging form.

In another preferred aspect, the delivery system in the treatment method of the present invention can be traditional water and oil emulsions, suspensions, colloids, microemulsions, clear solutions, suspensions of nanoparticles, emulsions of nanoparticles, or anhydrous compositions.

Additional ingredients or agents can also be included in the treatment method of the present invention, which can be selected from, but not limited to skin penetration enhancers, skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, solubilizers, anesthetics, colorants, perfumes, preservatives, seeds, broken seed nut shells, silica, clays, beads, luffa particles, polyethylene balls, mica, pH adjusters, processing aids, and combinations thereof.

The skin penetration enhancing agent is selected from methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof. Additional skin penetration agents suitable for use are included in a prior disclosure by the present inventor (U.S. patent application Ser. No. 20060110415), which is included in its entirety herein.

In another preferred aspect, in the treatment method of the present invention one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and phospholipids can be included.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948,1995 which is herein incorporated by reference.

The cosmetically acceptable carriers of this invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metals and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable carriers of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecylbenzene sulfonate.

Amphoteric surface-active agents that may be used in the cosmetically acceptable carriers of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable carriers of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyldimonium chloride, Dihydrogenated tallow benzylmonium chloride, disoyadimonium chloride, Ditallowedimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowedimonium chloride, myristalkonium chloride, (PEG=Polyethylene glycol) PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable carriers of the present invention may include long chain fatty amines from about C.sub.10 to C.sub.22 and their derivatives. Specific examples include dipalmitylamine, lauramidopropyldimethylamine, and stearamidopropyl dimethylamine. The cosmetically acceptable carriers of this invention may also include fatty alcohols (typically monohydric alcohols), ethoxylated fatty alcohols, and di-tail phospholipids, which can be used to stabilize emulsion or dispersion forms of the cosmetically acceptable carriers. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof.

Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable carriers of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable carrier of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable carrier of this invention are typically used in an amount from about 0.1 to 50 percent by weight, preferably from about 0.5 to about 40 percent by weight, more preferably from about 1 to about 20 percent by weight.

The cosmetically acceptable carrier of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable carrier of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable carrier of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The carriers of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane end blocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable carriers of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane-200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable carriers of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in the carriers of the present invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable carriers of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the trade name Permethyl 101A, available from Presperse, South Plainfield, N.J., USA. Another example of a preferred volatile hydrocarbon is C.sub.12 to C.sub.14 isoparaffin, under the trade name Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable carriers of this invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, and quaternary derivatives of guar. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable carrier of this invention at a concentration of from 0.1 to 10 weight percent, preferably from 0.2 to 6 weight percent and most preferably from 0.2 to 5 weight percent.

The cosmetically acceptable carrier of this invention may include one or more rheological modifiers. The rheological modifiers that can be used in this invention include high molecular weight crosslinked homopolymers of acrylic acid, and Acrylates/C10-30 Alkyl Acrylate Crosspolymer, such as the Carbopol and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropyltrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, poly(ethylene oxide) based thickeners, sodium carbomer, and mixtures thereof. When used, the rheology modifiers are preferably included in the cosmetically acceptable carrier of this invention at a concentration of from 0.01 to 12 weight percent, preferably from 0.05 to 10 weight percent and most preferably from 0.1 to 6 weight percent.

The cosmetically acceptable carrier of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 20 weight percent, preferably 0.5 to 10 weight percent and most preferably from 1.0 to 5.0 weight percent of the cosmetically acceptable carrier.

The cosmetically acceptable carrier of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl para-aminobenzoate (PABA), diethanolamine p-methoxy cinnamate, ethyl 4-bis (hydroxypropyl) aminobenzoate, 2-ethylhexy 1-2-cyano-3,3-diphenylacrylate, homomethyl salicylate, glyceryl aminobenzoate, dihydroxyacetone, octyl dimethyl PABA, 2-phenylbenzimidazole-5-sulfonic acid, triethanolamine salicylate, zinc oxide, zinc zeolite, titanium zeolite, and titanium oxide, and mixtures thereof. The amount of sunscreen used in the cosmetically acceptable carrier of this invention will vary depending on the specific UV absorption wavelength(s) of the specific sunscreen active(s) used and can be from 0.1 to 10 percent by weight, from 2 to 8 percent by weight.

The cosmetically acceptable carrier of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis(hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable carrier of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The cosmetically acceptable carrier of this invention can be presented in various forms. Examples of such forms include, but are not limited a solution, liquid, cream, emulsion, dispersion, gel, thickening lotion.

The cosmetically acceptable carrier of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerin, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture from 0.1 to 70 percent by weight, relative to the weight of the total composition.

The cosmetically acceptable carrier of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable carrier of this invention also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

The carriers of this invention for treating skin include leave-on or rinse-off forms such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, said carriers may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The carriers of this invention may be prepared as oil-in-water, water-in-oil emulsions, triple emulsions, or dispersions. Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

The carriers of this invention may be a shampoo, which may contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The carriers of this invention may be a shampoo for washing hair, which can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

The carriers of this invention may consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the carriers are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acrylnitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The carriers of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonite, silica, talc, zinc oxide or mixtures of these ingredients.

The carriers of this invention, in the form of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The carriers of this invention as solution or emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The carriers of this invention as suspensions may comprise liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminum metahydroxide, bentonite, agar and tragacanth or mixtures of these ingredients.

The carriers of this invention may contain additional antioxidants, which are selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The carriers of this invention may contain the blood micro-circulation improvement ingredients which are selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The carriers of this invention may include the anti-inflammatory agents, which are selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

### EXAMPLES

The following examples illustrate presently preferred practice thereof. As illustrations they are not intended to limit the scope of the invention. All quantities are in weight percent. C is temperature in degree Celsius.

### Example 1

### Preparation of a Carrier

The carrier or base used in Examples 2 to 4 can be of any suitable composition. A typical example of such a carrier base as a lotion is illustrated below. Ingredients (1) Purified Water 82.7994 (2) Glyceryl Stearate 1.0 (3) Phenoxyethanol 0.9 (4) Glycerin 3.8 (5) Mango oil 0.0001 (6) Pyridoxine 0.0001 (7) Aloe vera 0.0001 (8) Ascorbyl Palmitate 0.0001 (9) Methyl Paraben 0.2 (10) Propyl Paraben 0.1 (11) PGMS-SE 2.0 (12) Stearic Acid 3.0 (13) Cetyl Alcohol 1.2 (14) Carbomer 0.0001 (15) Botanical Extracts 5.0. Procedure. Mix (1) to (14) and heat at 75 to 85 C. Cool to 45 to 55 C and add all other ingredients. Mix and cool to room temperature.

### Example 2

### The Method of Topical Treatment with Zinc Zeolite—Hydroxy Acid Clathrate Complex for Acne

The following steps are performed for this method of topical treatment. (1) The zinc zeolite salicylate 25.0 and carrier lotion 75.0 are mixed together. (2) The mixture is applied topically in the amount and frequency necessary to achieve desired treatment. (3) A mechanical device is used to apply the complex. (4) After the application, the complex is left on skin for several hours before washing.

### Example 3

### The Method of Topical Treatment with Zinc Zeolite—Tyrosinase Inhibitor Clathrate Complex for Treating a Combination of Skin Wrinkles, Darkened Skin, and Skin Damage from UV

The following steps are performed for this method of topical treatment. (1) The zinc zeolite magnolol 2.0, zinc zeolite lactate 5.0, zinc zeolite hydroquinone 2.0, and carrier lotion 91.0, are mixed together. (2) The mixture is applied manually in the amount and frequency necessary to achieve desired treatment. (3) After the application, the complex is left on skin for several hours before washing.

### Example 4

### The Method of Topical Treatment with Zinc Zeolite—Matrix Metalloprotease Inhibitor—Zinc Zeolite—Lignan Clathrate Complex and a Penetration Enhancing Agent for Treatment of Diaper Rash, Chafed Skin, and Dry Skin.

The following steps are performed for this method of topical treatment. (1) The zinc zeolite phloridzin 2.0, zinc zeolite salicylate 5.0, zinc zeolite sesamin 2.0, triethyl citrate 5.0 (penetration enhancing agent) and carrier lotion 86.0, are mixed together. (2) The mixture is applied on a diaper, which is then placed on afflicted area of skin. (3) After the placement of said diaper on skin, it is left on skin for several hours before removing or washing, or on as needed basis to replace any soiled diaper.

### Example 5

### The Method of Topical Treatment with Zinc Zeolite—Glycol Clathrate Complex for Body Odor Treatment

The following steps are performed for this method of topical treatment. (i) A solid stick carrier base is first prepared as follows. Ingredients (1) Sodium Stearate 8.0 (2) Propylene Glycol 7.5 (3) PEG-47.5 (4) Cyclomethicone 40.0 (5) Isostearyl Alcohol 19.5 (6) PPG-10 Cetyl Ether 10.0 (7) Water 7.5. Procedure. Mix (1) to (3) and heat at 70 to 80 C. Cool to 60 to 65 C and add all other ingredients. (ii) Zinc zeolite ethylhexylglycerin clathrate is then made by mixing (1) zinc zeolite 30.0 (2) Ethylhexylglycerin 15.0 and (3) Methylpropanediol 55.0 at room temperature. (iii) The above carrier base 75.0 and Zinc zeolite ethylhexylglycerin clathrate 25.0 (from step (ii) above) are mixed at 60 to 65 C. This mixture is poured into a plastic deodorant stick packaging and cooled to room temperature until it turns into a solid stick. (iv) The solid stick thus obtained is applied manually in the amount and frequency necessary to achieve the desired body odor treatment.

1. A method for treating skin condition with certain clathrate complexes of zinc zeolite, wherein (i) said complex is applied topically at a desired site in a sufficient quantity, and (ii) wherein said application having been done either by a manual or a mechanical method, or a combination thereof, and (iii) wherein said topical application causes the desired treatment of said skin condition.

2. A method according to claim 1, wherein said skin condition is selected from the group consisting of acne, skin wrinkles, age spots, pimples, scars, skin rash including diaper rash, dry skin, scalp dandruff, scalp dry skin, darkened skin, skin discoloration, broken skin, chafed skin, sunburn, skin damage from UV, skin irritation, body odor, or a combination thereof.

3. A method according to claim 1, wherein said clathrate complex of zinc zeolite is of the chemical composition consisting of zinc zeolite and a non-antibiotic organic compound or agent.

4. A method according to claim 1, wherein a carrier is included.

5. A method according to claim 2, wherein said skin condition is acne.

6. A method according to claim 2, wherein said skin condition is body odor.

7. A method according to claim 2, wherein said skin condition is age spot.

8. A method according to claim 2, wherein said skin condition is a combination of darkened skin and skin discoloration.

9. A method according to claim 2, wherein said skin condition is a combination of scalp dandruff and scalp dry skin.

10. A method according to claim 2, wherein said skin condition is a combination of skin wrinkles, darkened skin, and skin damage from UV.

11. A method according to claim 2, wherein said skin condition is a combination of diaper rash, chafed skin, and dry skin.

12. A method according to claim 3, wherein said clathrate complex of zinc zeolite is further selected from the group consisting of zinc zeolite hydroxy acids, zinc zeolite hydroxy acid esters, zinc zeolite hydroxy acid salts, zinc zeolite organic peroxides, zinc zeolite organic hydroperoxides, zinc zeolite lignans, zinc zeolite enzyme inhibitors, zinc zeolite vitamins, zinc zeolite hormones, zinc zeolite peptides, zinc zeolite polyphenols, zinc zeolite stilbenes, zinc zeolite alkaloids, zinc zeolite plant extracts, zinc zeolite polyglycols, zinc zeolite sunscreens, zinc zeolite saponins, zinc zeolite anesthetics, zinc zeolite saponins and sapogenins, or combinations thereof

13. A method according to claim 4, wherein said carrier is further selected from the group consisting of a lotion, cream, gel, spray, thin liquid, body splash, mask, serum, solid cosmetic stick, lip balm, shampoo, liquid soap, bar soap, bath oil, paste, salve, powder, collodion, impregnated patch, impregnated strip, skin surface implant, skin penetration enhancing agent, diaper, anhydrous composition, or a combination thereof.

14. A method according to claim 12, wherein said zinc zeolite stilbenes is zinc zeolite resveratrol.

15. A method according to claim 12, wherein said zinc zeolite hydroxy acid is zinc zeolite salicylate.

16. A method according to claim 12, wherein said zinc zeolite enzyme inhibitor is zinc zeolite hydroquinone.

17. A method according to claim 12, wherein said zinc zeolite organic peroxides is zinc zeolite benzoyl peroxide.

18. A method according to claim 13, wherein said carrier is a lotion.

19. A method according to claim 13, wherein said carrier includes additional components selected from the group consisting of skin cleansers, surfactants, skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, anesthetics, colorants, perfumes, preservatives, seeds, broken seed nut shells, silica, clays, beads, luffa particles, polyethylene balls, mica, pH adjusters, processing aids, or combinations thereof.

20. A method according to claim 19, wherein said skin penetration enhancer is triethyl citrate.
